(19) United States
(12) Patent Application Publication
(10) Pub. No.: US 2006/0270648 A1
(43) Pub. Date: Nov. 30, 2006

(54) USE OF POLYAMINES WITH ANTIBIOTICS

(76) Inventors: Chung-Dar Lu, Roswell, GA (US); Dong H. Kwon, Atlanta, GA (US)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(21) Appl. No.: 11/420,671

(22) Filed: May 26, 2006

Related U.S. Application Data
(60) Provisional application No. 60/685,274, filed on May 27, 2005.

(51) Int. Cl. A61K 31/545; A61K 31/43; A61K 31/13

(52) U.S. Cl. 514/192; 514/200; 514/673

(57) ABSTRACT

Compositions and methods for affecting antibiotic resistance, for improving the overall efficacy of antibiotics, and for killing bacteria are included herein involving the use of polyamines to sensitize bacteria and resistant bacterial strains to hydrophilic antibiotics and to reduce bacterial resistance to such antibiotics.

### STATEMENT OF RELATED APPLICATIONS

This application claims priority under 35 USC 119 on U.S. Provisional Patent Application No. 60/685,274 entitled “Use of Polyamines with Antibiotics” having a filing date of 27 May 2005, which is hereby incorporated by reference.

### BACKGROUND OF THE INVENTION

1. Technical Field

This invention is related generally to the field of pharmaceutical treatments for bacterial infections and the field of disinfecting agents. More particularly, this invention is directed to compositions and methods for affecting antibiotic resistance, for improving the overall efficacy of antibiotics, and for killing bacteria.

2. Prior Art

Antibiotics and other antimicrobials have been widely used to save countless lives and to blunt serious complications of many diseases and infections. After more than 50 years of widespread use, many antimicrobials are becoming less effective against bacteria, in part because of bacterial resistance. Resistant bacteria have developed methods to circumvent the effects of antibiotics. Specifically, the overuse and widespread use of antibiotics is thought to have facilitated the evolutionary adaptations that enable bacteria to survive the most effective drugs. Antimicrobial resistance provides a survival benefit to microbes and makes it harder to eliminate infections.

The increasing incidence of bacterial resistance has increased the risk of acquiring more serious infections in a hospital or other setting. For example, diseases such as tuberculosis, gonorrhea, malaria, and childhood ear infections are now more difficult to treat as the pathogens causing these diseases have become more resistant to traditional antibiotics. Drug resistance is an especially difficult problem for hospitals harboring critically ill patients who are less able to fight off infections without the help of more powerful or more toxic antibiotics. Unfortunately, this worsens the problem by producing bacteria with greater abilities to survive even in the presence of the strongest known antibiotics. These even stronger drug-resistant bacteria continue to prey on vulnerable hospital patients. As a result of such infections, antibiotic resistance can result in longer hospital stays, higher mortality and increased health care costs.

Accordingly, there is always a need for compositions and methods for improving the biological activity of antibiotics. There is also a need for such compositions and methods for more effectively treating bacteria resistance to antibiotics. It is to these needs, among others, that the present invention is directed.

### BRIEF DESCRIPTION OF THE INVENTION

Briefly, this invention provides methods and compositions for improving the efficacy of antimicrobial agents and disinfectants. Exemplary methods and compositions for improving the efficacy of antibiotics against bacteria and resistant strains thereof involve the administration of polyamines to sensitize bacteria or resistant strains of bacteria to antibiotics and the administration of antibiotics to kill or inhibit the bacteria. The general principles of the present invention provide methods that can greatly improve the antimicrobial effects of antibiotics and disinfecting agents.

This invention generally includes a treatment of bacterial infections in a subject by administering an effective amount of polyamines alone or in combination with antibiotics. In one illustrative treatment, polyamines and antibiotics can be co-administered to subjects to induce an appropriate level of each by exogenous administration such that the polyamines sensitize the bacteria to the antibiotics and the antibiotics are better able to kill or inhibit the bacteria. In another illustrative treatment, the polyamines can be administered to subjects prior or subsequent to a course of treatment with antibiotics, so as to sensitize the resistant strains of bacteria to the antibiotics. In yet another illustrative treatment, the subject can be treated with antibiotics for a period of time prior to the administration of polyamines, which then can be administered to sensitize the bacteria or resistant strains of bacteria to the antibiotics. Thus, the polyamines can be administered to a subject concurrently with the antibiotics, prior to the antibiotics, and/or subsequent to the antibiotics.

The overall composition of this invention includes a therapeutically effective amount of the polyamines and antibiotics. The specific amount(s) of polyamines and/or antibiotics can be dependant on the antibiotics used, the polyamines used, the disease or infection to be treated, and the duration of the treatment. In general, the dosage amount can vary with such factors. A preferred dosage is the lowest dose of the polyamines and/or antibiotics that is therapeutic effective. The dosage can be determined by an attending physician or veterinarian within the context of sound medical judgment. Effective dosage forms, modes of administration and dosage amounts of polyamines and of antibiotics can be determined empirically, and making such determinations is within the skill of the art.

One advantage of this invention is that it can allow lower concentrations of some antibiotics to achieve therapeutic effectiveness. This present invention may also provide quality of life benefits due to, for example, decreased duration of therapy, reduced stay in intensive care units or overall in the hospital or clinic, and the concomitant reduced risk of serious nosocomial (hospital-acquired) infections.

This invention will become more apparent to those of ordinary skill in the relevant art when the following detailed description of the preferred embodiments is read in conjunction with the appended drawings.

### BRIEF DESCRIPTION OF THE FIGURES

FIG. 1 is a table depicting minimum inhibitory values of P. aeruginosa in the presence of various concentrations of polyamines, chemicals, and/or antibotics.

FIG. 2 is a table depicting the susceptibility of clinical isolates of P. aeruginosa to combinations of various antibiotics and polyamines.

FIG. 3 is a table depicting the susceptibility of strains of Escherichia. coli and Salmonella typhimurium to combinations of ciprofloxacin and various polyamines.

FIG. 4 graphically shows time killing assays of P. aeruginosa PAO1 in human serum.

FIG. 5 graphically shows that the β-lactamase activity of various cells decreases when treated with the combination of antibiotics and polyamines.

FIG. 6 graphically shows the β-lactamase activity of cells treated with disrupting agents.

FIG. 7 graphically shows that only a very low level of β-lactamase activity can be detected following a treatment with a combination of antibiotics and various polyamines.

FIG. 8 is a table depicting susceptibility of strains of Escherichia. coli strains to combinations of ciprofloxacin and various polyamines.

### DETAILED DESCRIPTION OF THE PREFERRED EMBODIMENTS

Preferred embodiments of this invention include compositions and methods for increasing the efficacy of antibiotics and other antimicrobials. Exemplary methods and compositions of this invention for improving the efficacy of antibiotics against bacteria and resistant strains thereof involve the administration of polyamines to sensitize the bacteria or resistant strains of bacteria to antibiotics and the administration of the antibiotics to kill or inhibit the bacteria. The general principles of the present invention are defined herein and provide methods that can greatly improve the antimicrobial effects of antibiotics and disinfecting agents.

More particularly, the administration or co-administration of various polyamines with antibiotics, particularly hydrophilic antibiotics, unexpectedly has been found to reduce bacterial resistance to such antibiotics, to kill resistant stains of bacteria, and to improve the efficacy of such antibiotics. As previous research has indicated that natural polyamines (e.g. cadaverine, spermidine, and spermine) can not sensitize enteric bacteria to hydrophobic antibiotics and that the natural polyamines have neither bactericidal nor sensitizing activity, the prior art has not focused on the uses of polyamines, particularly natural polyamines, to sensitize bacteria to antibiotics. After experimenting with various antibiotics (including hydrophilic antibiotics) and polyamines, it has been discovered that polyamines can sensitize bacteria, such as Pseudomonas aeruginosa, Escherichia coli, bacillus subtilis, and Staphylococcus aureus, to antibiotics and disinfecting agents.

One illustrative embodiment of this invention includes a treatment of bacterial infections in a subject by administering an effective amount of polyamines alone or in combination with antibiotics. In one illustrative treatment, polyamines and antibiotics can be co-administered to subjects to induce an appropriate level of each by exogenous administration such that the polyamines sensitize the bacteria to the antibiotics and the antibiotics are better able to kill or inhibit the bacteria. In another illustrative treatment, the polyamines can be administered to subjects prior or subsequent to a course of treatment with antibiotics, so as to sensitize the resistant strains of bacteria to the antibiotics. In yet another illustrative treatment, the subject can be treated with antibiotics for a period of time prior to the administration of polyamines, which then can be administered to sensitize the bacteria or resistant bacteria to the antibiotics. Thus, the polyamines can be administered to a subject concurrently with the antibiotics, prior to the antibiotics, and/or subsequent to the antibiotics.

For example, if a course of antibiotics is not proving effective enough, then a course of polyamines can be administered to sensitize the bacteria and increase the efficacy of the antibiotics. Similarly, a course of polyamines can be administered subsequent to a course of antibiotics simply to increase the efficacy of the antibiotics. For another example, a course of polyamines can be administered concurrently with a course of antibiotics to increase the efficacy of the antibiotics. Similarly, a course of polyamines can be administered concurrently with a course of an antibiotic generally considered less effective so as to increase the effectiveness of the antibiotic in cases where the subject may be allergic or otherwise sensitive to a different, medically preferred antibiotic. For yet another example, a course of polyamines can be administered prior to the administration of a course of antibiotics to pre-sensitize the bacteria to the antibiotics.

The general overall composition of this invention includes a therapeutically effective amount of the selected polyamines and antibiotics. The specific amount(s) of polyamines and/or antibiotics can be dependant on the antibiotics used, the polyamines used, the disease or infection to be treated (including the severity of bacterial infection or resistance thereof), the patient mass and condition, the rate of excretion of the composition, and the duration of the treatment. In general, the dosage amount can vary with such factors. A preferred dosage is the lowest dose of the polyamines and/or antibiotics that is therapeutically effective. The dosage can be determined by an attending physician or veterinarian within the context of sound medical judgment. Effective dosage forms, modes of administration and dosage amounts of polyamines and of antibiotics can be determined empirically, and making such determinations is within the ordinary skill of the art.

Because the use of some antibiotics is limited by their systemic toxicity or prohibitive cost, lowering the concentration of antibiotics required for therapeutic effectiveness reduces toxicity and/or cost of treatment, and thus allows wider use of the antibiotic and/or the use of alternative antibiotics. For example, administration of polyamines with antibiotics can circumvent the side effects of certain antibiotics and/or antimicrobial agents by substantially reducing the dosages of therapeutic agents comprising the antibiotics and/or antimicrobial agents. The present invention also may provide quality of life benefits due to, for example, decreased duration of therapy, reduced stay in intensive care units or overall in the hospital, and the concomitant reduced risk of serious nosocomial (hospital-acquired) infections.

Polyamines suitable for use with the present invention include natural polyamines, preferably those that are non-toxic natural compounds and/or exist at high levels in ordinary animal and/or human bodies. The polyamines selected for use with preferred embodiment of this invention are natural polyamines. Such natural polyamines include, for example, cadaverine, putrescine, spermidine, spermine, nor-spermidine, and nor-spermine. More preferred polyamines include spermine and spermidine as these polyamines were found to be more sensitizing to bacteria than putrescine and cadeverine. Artificial analogs and other polyamines are suitable with this invention and are available without undue experimentation.

An illustrative polyamine that can be used with this invention is spermine as this polyamine has shown the highest efficacy in reducing bacterial resistance to antibiotics and in improving the efficacy of antibiotics. As shown in the Examples described herein, this polyamine can sensitize resistant bacteria and together with various antibiotics can be effective in treating bacterial infections. As this polyamine also is non-toxic to humans and naturally found in a human body, this polyamine can be administered safely to human patients. The results disclosed in the examples can be extrapolated to other polyamines

Antibiotics suitable with this invention include substances, produced synthetically or naturally, that can inhibit the growth of or kill microorganisms. Such antibiotics can include β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. Exemplary β-lactam antibiotics include ampicillin, aziocillin, aztreonam, carbenicillin, cefoperazone, ceftriaxone, cephaloridine, cephalothin, cloxacillin, moxalactam, penicillin G, piperacillin, and ticarcillin. Other antibiotics including cation peptides also are suitable with this invention.

Antibiotics for use with this invention further include compositions or treatments that include one or more antibacterial agents or antibiotics. For example, first and second antibiotics administered in a series (one after the other) or in parallel (at the same time or as a combination) can be employed with this invention and are included in the term antibiotics. In one embodiment, the combination of first and second antibiotics may be, for example, a penicillin and an aminoglycoside, such as gentamycin or vancomycin. In a preferred embodiment, combinations of antibiotics include at least two antibiotics that are different from each other and each antibiotic preferably is from a different class of antibiotic.

As is evident to those with ordinary skill in the art, the polyamines and/or antibiotics compositions can be administered using known and future developed medical methods. The compositions can be formulated for administration by a variety of routes of administration. In preferred embodiments, the antibiotic product is formulated in a manner suitable for oral administration, which can include each of the dosage forms as a pellet or a particle, with a pellet or particle then being formed into a unitary pharmaceutical product, for example, in a capsule, or embedded in a tablet, or suspended in a liquid for oral administration.

Further, in formulating an oral delivery system, each of the dosage forms of the antibiotic and polyamine product may be formulated as a tablet, with each of the tablets being put into a capsule to produce a unitary antibiotic product. Thus, for example, antibiotic products may include a first dosage form in the form of a tablet that is an immediate release tablet, and also may include at least one additional tablet that provides for a delayed release of the antibiotic, whereby the antibiotics or polyamines released from each of the tablets is released at different times, with the total antibiotic released from the antibiotic product being achieved in a desired period of time. As known in the art, with respect to delayed release, the time of release can be controlled by the concentration of antibiotics in the coating and/or the thickness of the coating.

In this manner, the antibiotic portion and the polyamine portion can be released concurrently or at different times. For example, the quick release (first to release) tablet can be either the antibiotic or the polyamine and the slower release (second to release) tablet can be the polyamine or antibiotic, respectively. Further, slower (third and greater to release) tablets also can be included comprising antibiotics and/or polyamines, as desired. This type of release system allows the medical practitioner to decide whether to release the antibiotic or the polyamine first. Alternatively, the antibiotic and the polyamine can be combined in a single tablet, or can be separate but in similar time releasing tablets.

Further, the polyamines or the combination of polyamines and antibiotics also may be designed for use in ointments and other topical applications of disinfectants. For example, the combination of polyamines with antibiotics can be especially effective as antibacterial washes. In one example, the topical administration can be applied in at least two different dosage forms, each of which contain antibiotics or polyamines, and may be formulated for topical administration by including such dosage forms in an oil-in-water emulsion, or a water-in-oil emulsion. In such a formulation, the immediate release dosage form is in the continuous phase, and the delayed release dosage form is in the discontinuous phase. The formulation also may be produced in a manner for delivery of multiple dosage forms. For example, there may be provided an oil-in-water-in-oil emulsion, with oil being a continuous phase that contains the immediate release component and water dispersed in the oil containing a first delayed release dosage form. The ointment can be in the form of a cream or emulsion, or other dissolvable dosage form similar to those used for topical administration. Thickeners such as synthetic polymers, fatty acids, fatty acid salts and esters, fatty alcohols, modified celluloses or modified mineral materials also can be employed with liquid carriers to form spreadable pastes, gels, ointments, soaps, and the like, for application directly to the skin of the user.

Sterile injectable solutions also can be prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions. The solution can be combinations of antibiotics and polyamines. Alternatively, separate injections of antibiotics and of polyamines can be administered in the desired sequence. Such solutions alternatively can comprise microspheres or nanospheres of antibiotics and of polyamines, which microspheres or nanospheres dissolve of activate within the body at predetermined rates.

In addition to in vivo applications, it also is contemplated that suitable polyamines could be used with antibiotics under in vitro conditions. For example, polyamines could be used with antibiotics in the food industry to keep foods fresher longer. In such uses, the polyamines would help the antibiotics inhibit bacteria that cause the rotting of the food products, thus increasing shelf life. For another example, polyamines could be used with bloods, cells, or tissues to keep such matter low or free of bacteria. In such uses, the polyamines would help the antibiotics inhibit bacteria that cause the tainting of the bloods, cells, or tissues, thus increasing the effective useful life of the bloods, cells, or tissues.

The mechanism by which polyamines act with antibiotics or confer resistance is not yet completely understood and the effect may not extend to all polyamines. It is believed that the polyamines can act by influencing the e. flux pump. Further, it is believed that the polyamines can act by influencing penicillin binding proteins. The preferred antibiotics are those that work through one of these mechanisms. However, the precise mechanism by which polyamines are able to effect treatment of drug resistant infections and improve the efficacy of antibiotics is not critical.

### EXAMPLES

The data and examples in this section have been included to establish that the efficiency of antibiotics can be enhanced by the administration of or combination with polyamines. More particularly, this invention is further illustrated by reference to the following non-limiting examples.

General Materials and Methods

Experimental Growth Conditions

Various strains of bacteria, including P. aeruginosa and e. coli were grown on Luria-Bertani (LB) agar/broth or Mueller-Hinton (MH) agar/broth. A growth curve was determined by growing the bacteria in the presence of various chemicals, polyamines and/or antibiotics. The inoculated cells then were cultured in an air-temperature controlled incubator. Aliquots were withdrawn at every hour and the optical density was measured at 600 nm. The antibiotics, chemicals, and/or polyamines were dissolved in double distilled water or solvent and thereafter filtered through a 0.4 μm disposable membranes.

Time Killing Assay

Time-killing assays were performed for various bacteria samples. Briefly, the tested bacteria were grown to mid-log phase in Mueller-Hinton broth media. The cells were diluted gently in a sodium phosphate buffer containing a polyamine (e.g. spermine) at room temperature. As a control group, cells were diluted in a sodium phosphate without any additional compounds. Aliquots were withdrawn at specific time intervals and spread on LB agar plates and incubated overnight at 37 C. The percent of colonies that survived were determined relative to the control.

Minimum Inhibitory Concentration (MIC) Values

The MIC values of the strains associated with various combinations of polyamines and antibiotics were determined using standard two-fold agar dilutions techniques and broth dilutions techniques in Mueller-Hinton broth. The lowest concentration of drug with no visible turbidity was deemed the MIC. Antibiotic diffusion assay also was performed to verify the results from agar dilution and broth dilution techniques.

β-Lactamase Assay

The β-lactamase activity was determined by a spectrophotometric method using nitrocefin as a substrate. After the β-lactamase was extracted from the cells, the β-lactamase activity was monitored by absorbance changes in wavelength at 486 nm with a molar extinction coefficient of 20500. The extent of hydrolysis was a reflection of the amount of enzyme remaining uninhibited.

Construction of lacZ::ampC Promoter Fusion and β-Galactosidase Assays

Genomic DNA was extracted from various bacteria, e.g., P. aeruginosa PAO1 and was used in amplifying the ampC (PA4110) promoter region with a PCR primer pair, 5′-ggaagtcctccagccgcggcag-3′/5′-ggcgtcctttgtcgttggctgcatgagaaa-3′ (500-bps fragment). The 500-bps PCR fragment was purified using spin columns and was inserted into a broad-host-range transcriptional fusion vector, pQF50, which resulted in plasmid pAU16R. The orientations and DNA sequences of the insert were confirmed by nucleotide sequencing.

The cultures harboring the pAU16R were diluted 100-fold in 20 mL of MH broth with or without spermidine (10 mM and 20 mM). The cultures were incubated at 350 rpm and at 37 C for 3 hours. After the antibiotic was was added in each culture to induce the ampC gene, the culture was incubated at the same condition for an additional hour. The cells were harvested by centrifugation, washed once, and resuspended in a phosphate buffer (pH 7.0). French pressure cell at 8,000 lb/in² was used to break the cells and soluble cell extracts were prepared for measurements of μ-galactosidase activity. The protein concentration was determined by the Bradford method using bovine serum albumin as the standard.

Outer Membrane Permeabilization Assays

An outer membrane permeabilization assay was performed by examining the release of the chromosomally encoded β-lactamase. Briefly, stationary phase cells were diluted 1:59 into 20 mL pre-warmed MMP broth and cultured in an incubator at 350 rpm and at 37 C for 5 hours. After carbenicillin (200 μg/mL) was added to induce β-lactamase activity, the culture was incubated for additional 5 hours. The cells were harvested by centrifugation at 5,000×g for 10 minutes and washed once with 0.05M sodium phosphate buffer at pH 7.2. The cell pellet was resuspended in 5 mL of the same buffer and divided into 1 mL aliquots. The outer membrane permeabilization assay was commenced by the addition of 1, 5, 10, 20 mM for EDTA, spermidine, spermine and arginine (as a control) to each aliquot. The assay was also commenced by addition of 1, 5, 10, 20 μg/mL for PMBN to each aliquot. The reaction mixtures were incubated at room temperature for 5 minutes and immediately centrifuged at full speed (15000×g) for 20 minutes and the supernatant was used for β-lactamase sources. β-lactamase activity was determined as described above using 300 μL of the supernatant.

### Example 1

The combination of polyamines and various antibiotics was more effective against bacterial infections of P. aeruginosa than antibiotics alone. These results show that combining polyamines with certain antibiotics can inhibit bacterial growth and can have a synergistic effect in that combining polyamines with certain antibiotics improves the efficacy of antibiotics. Further, these results indicate that the concentration of the particular polyamine may be selected to completely inhibit bacterial resistance.

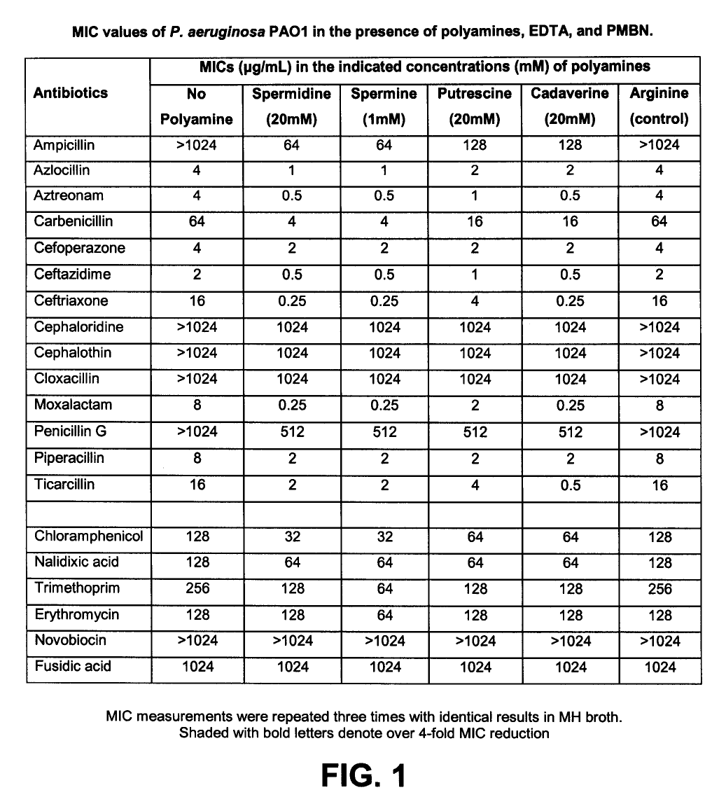

FIG. 1 shows that polyamines were able to sensitize a resistant strain of P. aeruginosa (P. aeurginosa PAO1). Summarily, 20 mM spermidine, putrescine, cadaverine and 1 mM of spermine were able to sensitize P. aeruginosa to 14 β-lactams, chloramphenicol, nalidixic acid, and trimethoprim. The MIC values for all β-lactam antibiotics, chloramphenicol, nalidixic acid, and trimethoprim were decreased significantly (2- to 64-fold) in the presence of all polyamines (spermidine, spermine, putrescine, and cadaverine). Spermine and spermidine appeared to have better sensitization effects than those of the putrescine and cadaverine at the concentrations used in this example. The MIC values to hydrophobic antibiotics (erythromycin, novobiocin, and fusidic acid) were identical between the presence and the absence of polyamines. Further, polyamines also sensitized the ciprofloxacin resistant strains to carbenicillin, chloramphenicol, and nalidixic acid.

As mutant strains with very high MIC values became sensitive to antibiotics in the presence of polyamines, this example shows P. aeruginosa can be more effectively treated by combining antibiotics, such as ciprofloxacin, β-lactams, chloramphenicol, nalidixic acid, and trimethoprim, with polyamines.

### Example 2

The addition of higher concentrations of polyamines can further improve the sensitization of the bacteria and the efficacy of the antibiotics. To further examine the effects of polyamines on MIC values of antibiotics, different concentrations of polyamines were used to determine MIC values to carbenicillin. Table 1 reveals that higher concentrations of polyamines decreased higher-levels of MIC values. The MIC values in this table were repeated three times and yield the same values. All of the MIC results, decreased or unchanged, were verified by antibiotic inhibition zone testing.

TABLE 1
MICs of carbenicillin against P. aeruginosa PAO1 in the presence of
different concentrations of polyamines.

| | MICs (μg/mL) to carbenicillin in the indicated | | | | | |
|---|---|---|---|---|---|---|
| | concentration of polyamines (mM) | | | | | |
| Compounds | 0 | 0.5 | 1 | 5 | 10 | 20 |
| Spermidine | 64 | 32 | 16 | 16 | 8 | 4 |
| Spermine | 64 | 16 | 4 | 4 | 4 | 4 |
| Putrescine | 64 | 64 | 32 | 32 | 32 | 16 |
| Cadaverine | 64 | 64 | 32 | 32 | 32 | 16 |

### Example 3

Resistant strains of P. aeruginosa (or clinical isolates) were sensitized by various combinations of antibiotics and polyamines. The MIC values of the reference strain P. aeruginosa PAO1 to carbenicillin, chloramphenicol, and nalidixic acid were confirmed as identical as the previous ones. In the absence of polyamines, the MIC values of the clinical isolates to carbenicillin and nalidixic acid were much higher than those of the reference strain PAO1 except for the strain T6268 for carbenicillin.

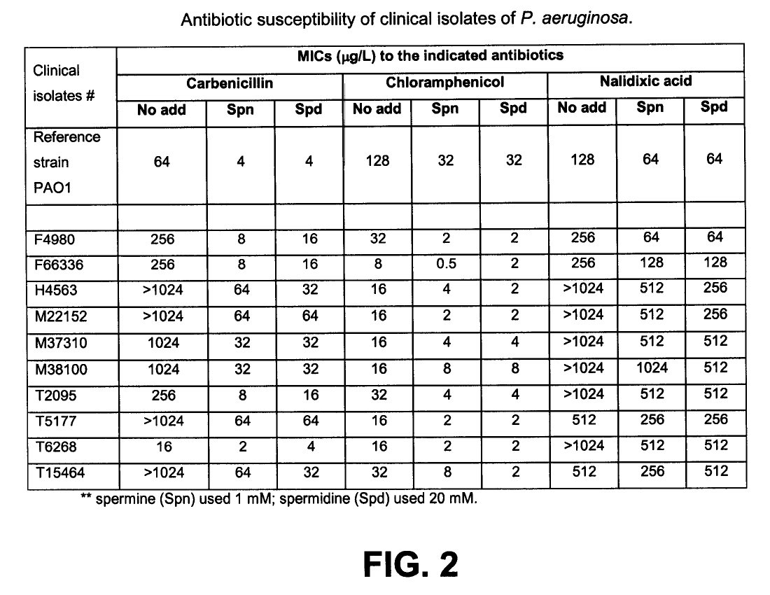

More particularly, as shown in FIG. 2, the MIC values of the clinical isolates to carbenicillin, chloramphenicol, and nalidixic acid were decreased from 2- to 32-fold in comparison to those from absence of polyamines. The reference strain PAO1 was surprisingly much higher levels of MIC to chloramphenicol than those of the clinical isolates and ranged from 4- to 16-fold (FIG. 2). The MIC values of the clinical isolates to ciprofloxacin, gentamicin, and polymyxin B were similar or increased up to 4-fold in the presence of polyamines. This example confirms that the combinations polyamines and antibiotics are able to lower the MIC values of different strains.

### Example 4

The combination of polyamines and antibiotics was able to sensitize strains of E. coli and Salmonella typhimuium. Various strains of E. coli (e.g. K10, K12, and C921-61 isolate from europathogenic patient) and S. typhimurium LT2 showed improved susceptibility to β-lactams (ampicillin, azlocillin, carbenicillin, oxacillin, penicillin G, piperacillin, and ticarcillin) and 7 other antibiotics (chloramphenicol, erythromycin, fusidic acid, kanamycin, novobiocin, spectinomycin, and tetracycline).

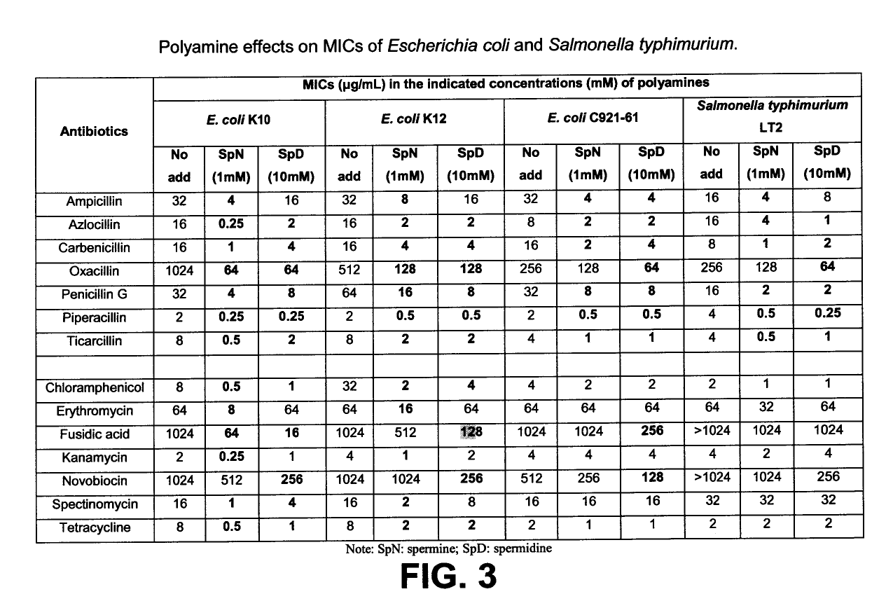

As shown in FIG. 3, E. coli strains and S. typhimurium were more than 4 fold more susceptible to most of the β-lactam antibiotics in the presence of polyamines compared to the absence of polyamines. Similarly, E. coli strains (K10 and K12) were also more than 4-fold more susceptible to most of the other antibiotics in the presence of polyamines. As such, this example shows that the combination of polyamines and antibiotics are able to lower the MIC values of different bacteria.

### Example 5

The combination of polyamines and antibiotics is able to sensitize methicillin-resistant Staphylococcus aureus (MRSA) to β-lactams. A clinical isolate of MRSA, Staphylococcus aureus Mu5, showed high level resistance to β-lactams and intermediate level resistance to vancomycin. More particularly, Table 2 shows that exogenous spermine (1 mM and less) makes Staphylococcus aureus Mu50 susceptible again to β-lactams (e.g. MIC of oxacillin decreased over 100-fold). Staphylococcus aureus Mu50 is known to express a high level of penicillin binding protein 2A encoded by mecA as the molecular mechanism of methicillin resistance. As glycopeptide antibiotics (e.g. vancomycin) were considered the last defense line for MRSA and as clinical application of vancomycin results in the emergence of vancomysin/methicillin double resistant strains, this example shows that the combination of polyamines and various antibiotics can be a treatment for these super-resistant bacteria.

TABLE 2
Effect of spermine on antibiotic susceptibility of methicillin resistant
Staphylococcus aureus (MRSA) Mu50.

| | |
|---|---|
| | MICs (μg/mL) in the |
| | presence or absence of |
| Antibiotics | spermine (1 mM) |

| (β-lactam antibiotics) | No add | Spermine |
|---|---|---|
| Ampicillin | ≧32 | 2 |
| Amoxicillin | 16 | 0.5 |
| Azlocillin | 16 | 0.5 |
| Carbenicillin | 256 | 4 |
| Cefoperazone | >512 | 16 |
| Cefotaxime | 128 | 1 |
| Ceftazidime | 256 | 4 |
| Ceftriaxone | >512 | 16 |
| Cephaloridine | 16 | 0.25 |
| Cephalothin | 32 | 4 |
| Cloxacillin | ≧256 | 1 |
| Imipenem | | |
| Oxacillin | ≧256 | 1 |
| Penicillin G | 16 | 0.25 |
| Piperacillin | 32 | 2 |
| Ticarcillin | 256 | 4 |
| Chloramphenicol | 8 | 2 |
| Ciprofloxacin | 64 | 32 |
| Norfioxacin | 128 | 64 |
| Polymyxin B | 128 | 64 |
| Tetracycline | 32 | 8 |

### Example 6

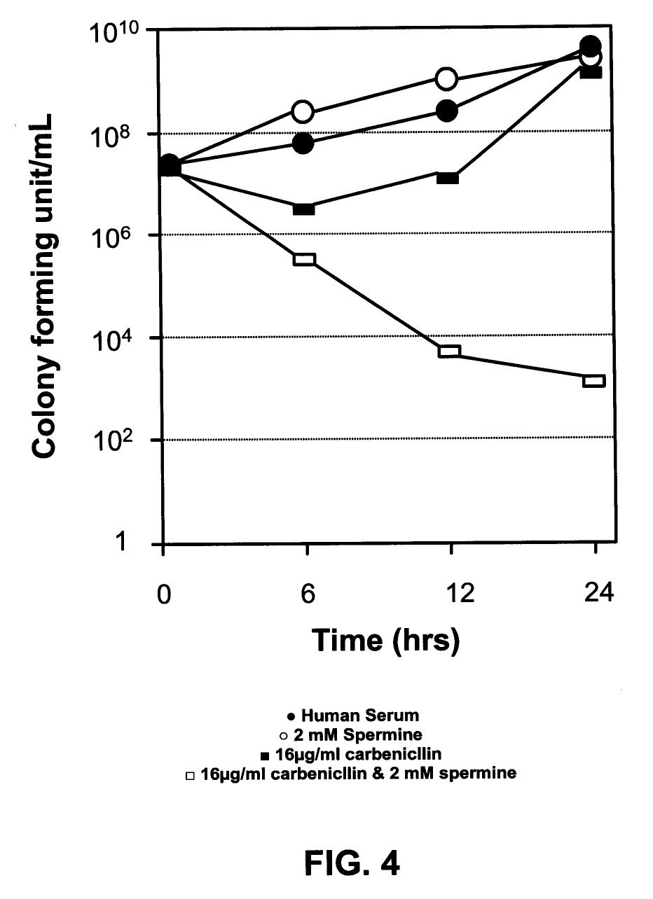

Polyamines were able to sensitize bacteria to antibiotics in human serum. FIG. 4 shows the results of polyamine-mediated β-lactam susceptibility testing in human serum. Polyamines alone in human serum were able to kill more than 99% of two E. coli strains (K10 and K12) within 24 hours without any exogenous antibiotics; however, P. aeruginosa PAO1 and S. typhimurium LT2 still grew well in the same condition (data not shown).

As shown in FIG. 4, the growth of P. aeruginosa PAO1 in human serum was not affected by the presence or absence of 2 mM spermine. Further, while the addition of 16 μg/mL carbenicillin alone into the serum caused an initial inhibition, this inhibition was followed by a full recovery of growth. However, more than 99% of the inoculants were killed with 24 hours when 2 mM spermine was added along with 16 μg/mL carbenicillin. Similar results also were obtained with S. typhimurium LT2 (data not shown).

These positive results in human serum strongly supports that combinations of polyamines and antibiotics could be effective in humans.

### Example 7

This example shows that cell polyamines do not change the outer membrane permeability or rupture the cell or outer membrane. Measurements of β-lactamase activities in cell-free filtrates or whole cells have been used to assess whether changes on the outer membrane barrier are associated with increased antibiotic susceptibility. When treated with polymyxin B, a cationic peptide antibiotic known to increase outer membrane permeability, significant activities of β-lactamase were detected in the whole cells when 100 μg/mL of this antibiotic was applied to the cell suspension

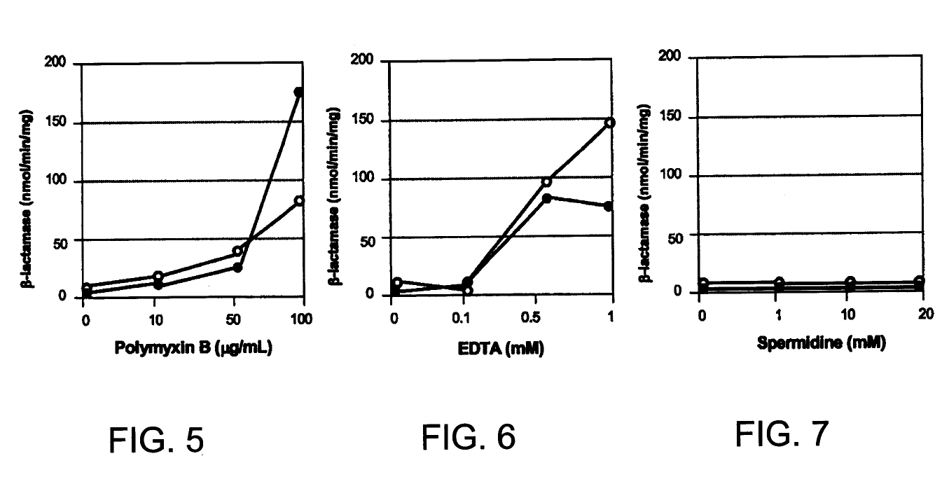

FIG. 5 shows that cells treated with polymyxin B show significant activities of β-lactamase in the whole cells when 100 μg/mL of this antibiotic was applied to the cell suspension. FIG. 6 shows that the release of periplasmic β-lactamase into the suspension solution as the result of outer membrane rupture can be detected following addition of 0.5 mM EDTA and increased with the addition of 1 mM of EDTA, and outer membrane permeability also was increased as evidenced by the activities detected from whole cells. FIG. 7 shows that only a very low level of β-lactamase activity was detected following the addition of 20 mM spermidine or spermine (data not shown). These results indicate that it is very unlikely that polyamines exert their effects by rupturing the outer cell membrane or by changing cell outer membrane permeability.

### Example 8

Polyamine-mediated antibiotic sensitization is not affected by the presence of divalent ions or salts. As previous research has shown that physiological concentrations of divalent ions or salt have affect on antibiotic susceptibility of some antibiotics, the combination of polyamines and antibiotics was tested in varying concentrations of divalent ions or salt affect.

As shown in Table 3, increased concentrations of divalent ions or salt had no significant effect in the sensitization of bacteria by polyamines. For example, it is shown in this example that that the combination of spermine and spermidine remained effective in the presence of 10 mM spermidine. Increased concentrations of divalent ions or salt had no significant effect on carbenicillin susceptibility in the presence of 10 mM spermidine. Similar results were also observed in Pseudomonas aeruginosa, Salmonella, typhimurium, and methicillin resistant Staphylococcus aureus Mu50 (MRSA).

TABLE 3
Effects of magnesium, calcium, and sodium chloride on carbenicillin
susceptibility of E. coli K10.

| | MICs (μg/mL) to carbenicillin in the | | |
|---|---|---|---|
| MHB medium | indicated concentrations of polyamines | | |
| supplemented | | | Spermidine |
| with | No addition | Spermine (1 mM) | (10 mM) |
| No addition | 16 | 1 | 4 |
| 1 mM MgSO₄ | 16 | 1 | 4 |
| 3 mM MgSO₄ | 16 | 4 | 4 |
| 1 mM CaCl₂ | 16 | 1 | 2 |
| 3 mM CaCl₂ | 16 | 4 | 4 |
| 100 mM NaCl | 16 | 4 | 4 |
| 150 mM NaCl | 16 | 4 | 4 |

### Example 9

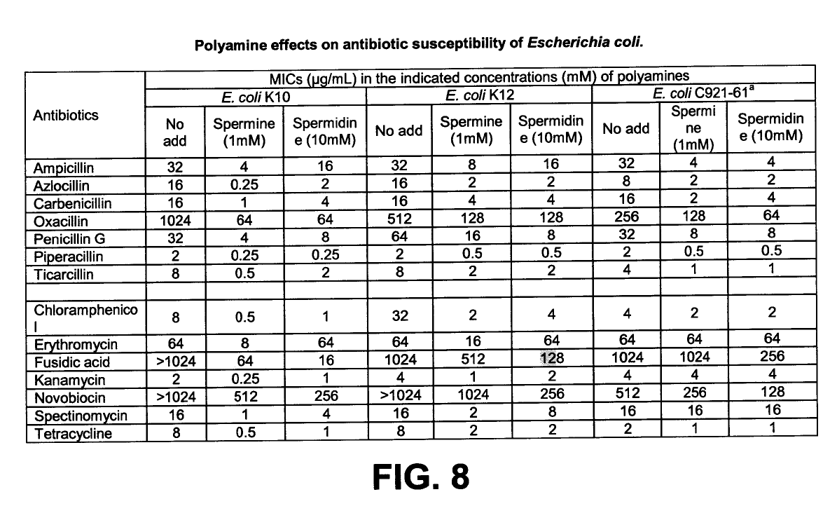

A combination of polyamines and various antibiotics provided increased efficacy against various strains of resistance e. coli. As shown in FIG. 8, polyamines increased antibiotic susceptibility of various resistant strains of e. coli to various antibiotics. As shown in FIG. 8, the MIC values were lowered by a factor of four with the addition of exogenous polyamines.

### Example 10

A combination of polyamines and various antibiotics provided increased efficacy against a resistant strain of Salmonella typhimurium. In this example, polyamines increased the antibiotic susceptibility of Salmonella typhimurium to various antibiotics. As shown in Table 4, the MIC values were lowered by a factor of four.

TABLE 4
Polyamine effects on antibiotic susceptibility of
Salmonella typhimurium LT2.

| | MICs (μg/mL) in the indicated concentrations | | |
|---|---|---|---|
| | (mM) of polyamines | | |
| | | Spermine | Spermidine |
| Antibioitcs | No add | (1 mM) | (20 mM) |
| Ampicillin | 16 | 4 | 8 |
| Azlocillin | 16 | 4 | 1 |
| Carbenicillin | 8 | 1 | 2 |
| Oxacillin | 256 | 128 | 64 |
| Penicillin G | 16 | 2 | 2 |
| Piperacillin | 4 | 0.5 | 0.25 |
| Ticarcillin | 4 | 0.5 | 1 |
| Chloramphenicol | 2 | 1 | 1 |
| Erythromycin | 64 | 32 | 64 |
| Fusidic acid | >1024 | 1024 | 1024 |
| Kanamycin | 4 | 2 | 4 |
| Novobiocin | >1024 | 1024 | 256 |
| Spectinomycin | 32 | 32 | 32 |
| Tetracycline | 2 | 2 | 2 |

### Example 11

A combination of polyamines and various antibiotics provided increased efficacy against a resistant strain of Staphylococcus aureus 700699. In this example, polyamines increased antibiotic susceptibility of Staphylococcus aureus 700699 to various antibiotics. As shown in Table 5, the MIC values were lowered by a factor of four.

TABLE 5
Polyamine effects on antibiotic susceptibility of
MRSA Staphylococcus aureus 700699.

| | MICs (μg/mL) in the absence or | |
|---|---|---|
| | presence of spermine | |
| Antibiotics | No add | Spermine (1 mM) |
| Ampicillin | ≧32 | 2 |
| Amoxicillin | 16 | 0.5 |
| Azlocillin | 16 | 0.5 |
| Aztreonam | >1024 | >256 |
| Carbenicillin | 256 | 4 |
| Cefoperazone | >512 | >4 |
| Cefotaxime | 128 | 1 |
| Ceftazidime | 256 | 4 |
| Ceftriaxone | >512 | 16 |
| Cephaloridine | 16 | 0.25 |
| Cephalosporin C | >512 | >128 |
| Cephalothin | 32 | 4 |
| Cloxacillin | >256 | 2 |
| Moxalactam | >512 | >32 |
| Oxacillin | ≧256 | 2 |
| Penicillin G | 16 | 0.25 |
| Piperacillin | 32 | 2 |
| Ticarcillin | 256 | 4 |
| Vancomycin | 4 | 4 |
| Chloramphenicol | 8 | 2 |
| Ciprofloxacin | 32 | 8 |
| Gentamicin | 256 | 256 |
| Kanamycin | >16 | >16 |
| Tetracycline | 32 | 4 |

The foregoing detailed description of the preferred embodiments and the appended figures have been presented only for illustrative and descriptive purposes and are not intended to be exhaustive and are not intended to limit the scope and spirit of the invention. The embodiments were selected and described to best explain the principles of the invention and its practical applications. One skilled in the art will recognize that many variations can be made to the invention disclosed in this specification without departing from the scope and spirit of the invention.

What is claimed is:

1. A method for improving the efficacy of antibiotics in a subject, comprising the steps of:
a) administering at least one polyamine to the subject; and
b) administering at least one antibiotic to the subject.

2. The method as claimed in claim 1, wherein the efficacy of the at least one antibiotic in combination with the at least one polyamine is greater than the efficacy of the at least one antibiotic alone.

3. The method as claimed in claim 1, wherein the amount of the at least one antibiotic needed to inhibit a bacterial infection in the subject is reduced by the administration of the at least one polyamine to the subject.

4. The method as claimed in claim 1, wherein the at least one antibiotic is hydrophilic.

5. The method as claimed in claim 1, wherein the at least one antibiotic is a β-lactam.

6. The method as claimed in claim 1, wherein the at least one antibiotic is selected from the group consisting of penicillin, cephalosphorins, monobactams, and combinations thereof.

7. The method as claimed in claim 1, wherein the at least one polyamine includes a naturally occurring polyamine.

8. The method as claimed in claim 1, wherein the at least one polyamine is selected from the group consisting of cadaverine, putrescine, spermidine, spermine, nor-spermidine, and nor-spermine, and combinations thereof.

9. The method as claimed in claim 1, wherein the at least one polyamine includes a chemically modified naturally occurring polyamine.

10. The method as claimed in claim 1, wherein the at least one polyamine includes a polyamine derived from putrescine.

11. The method as claimed in claim 1, wherein the efficacy of the at least one antibiotic in combination with the at least one polyamine has a greater efficacy against bacteria resistant than the at least one antibiotic alone.

12. The method as claimed in claim 1, wherein the at least one polyamine is administered prior to the at least one antibiotic.

13. The method as claimed in claim 1, wherein the at least one polyamine is administered subsequent to the at least one antibiotic.

14. The method as claimed in claim 1, wherein the at least one polyamine and the at least one antibiotic are concurrently administered to the subject.

15. The method as claimed in claim 1, wherein the at least one antibiotic comprises multiple antibiotic agents.

16. The method as claimed in claim 1, wherein the at least one polyamine is an artificial analog of a naturally occurring polyamine.

17. The method as claimed in claim 1, wherein the at least one antibiotic is administered in an oral dosage form.

18. The method as claimed in claim 1, wherein the at least one polyamine is administered in an oral dosage form.

19. A composition for treating bacterial inventions comprising:
a) at least one polyamine;
b) at least one antibiotic; and
c) a pharmaceutically acceptable carrier.

20. The composition as claimed in claim 19, wherein the at least one polyamine is derived from putrescine.

21. The composition as claimed in claim 19, wherein the at least one polyamine is selected from the group consisting of β-lactam antibiotics, aminoglycosides, macrolides, monobactams, rifamycins, tetracyclines, chloramphenicol, clindamycin, lincomycin, imipenem, fusidic acid, novobiocin, fosfomycin, fusidate sodium, neomycin, polymyxin, capreomycin, colistimethate, colistin, gramicidin, minocycline, doxycycline, vanomycin, bacitracin, kanamycin, nalidixic acid, and trimethoprim.

22. The composition as claimed in claim 19, wherein the at least one antibiotic is hydrophilic.

23. The composition as claimed in claim 19, wherein the at least one antibiotic is a β-lactam.

24. The composition as claimed in claim 19, wherein the at least one antibiotic includes a combination of different antibiotics.

25. The composition as claimed in claim 19, wherein the at least one antibiotic includes a combination of disparate class antibiotics.

26. The composition as claimed in claim 19, wherein the at least one antibiotic affect bacteria by affecting an efflux pump within the bacteria.

27. The composition as claimed in claim 19, wherein the at least one antibiotic affects penicillin binding proteins.
